(19) United States
(12) Patent Application Publication
(10) Pub. No.: US 2023/0143367 A1
(43) Pub. Date: May 11, 2023

(54) Elastic Makeup Powder Derived From Natural Inorganic Particulates And Method Of Preparing Same

(71) Applicant: SUNJIN BEAUTY SCIENCE CO., LTD., Seoul (KR)

(72) Inventors: Sung Ho LEE, Seoul (KR); Yeong Gi KIM, Seoul (KR); Bou Kun CHOI, Seoul (KR)

(21) Appl. No.: 17/914,941

(22) PCT Filed: Apr. 7, 2020
(86) PCT No.: PCT/KR2020/004695

§ 371 Date: Oct. 25, 2022

(30) Foreign Application Priority Data
Mar. 27, 2020 (KR) 10-2020-0037832

(51) Int. Cl. A61K 8/02; A61K 8/25; A61K 8/37; A61Q 1/12

(52) U.S. Cl. CPC A61K 8/022; A61K 8/25; A61K 8/375; A61Q 1/12; A61K 2800/622; A61K 2800/63; A61K 2800/805

(57) ABSTRACT

The present invention relates to an elastic makeup powder derived from natural inorganic particulates, and a method of preparing same, and more specifically, to an elastic natural makeup powder and a method of preparing same, the elastic natural makeup powder being characterized by comprising: natural inorganic particulates therein; and an elastic coating layer, which is dry-coated on the surface of the inorganic particulates and comprises (a) a triester of hydroxy fatty acids with glycerin, which his solid at room temperature, and a derivative thereof, or (b) a glyceryl fatty acid ester, which is solid at room temperature, and a derivative thereof. The elastic natural makeup powder of the present invention utilizes natural ingredients and thus, when applied to a cosmetic material, exhibits skin stability, is safe to the human body, exhibits excellent dispersibility, and can significantly improve the texture of the cosmetic material.

### FIELD

The present invention relates to an elastic makeup powder derived from natural inorganic particulates and a method of preparing the same, and more particularly, to an elastic makeup powder derived from natural inorganic particulates, which uses a natural material when applied to a cosmetic material, thereby having excellent skin stability, being safe to the humanbody, having excellent dispersibility and capable of significantly improving the texture of the cosmetic material.

### BACKGROUND

The content described below merely provides background information related to the present invention and does not constitute the prior art.

As makeup techniques have developed, various types of cosmetics are coming into use, and various synthetic materials as well as natural cosmetic materials are being applied to cosmetics.

In order to improve the texture of conventional cosmetics, makeup powders have been used in cosmetics, among which an elastic powder has been used for even better texture.

As an example, Korean Unexamined Patent Application Publication No. 10-2014-0026818 discloses that elastic powders such as PMMA, nylon powder, silicon elastomer and polyurethane are applied to cosmetic materials as spherical powders with elasticity.

However, recently, as serious problems regarding harmfulness to the human body and environmental pollution caused by synthetic powders have emerged, there has been a trend in various countries to restrict the use of synthetic powders that have been applied to conventional cosmetics, and particularly, in the case of elastic makeup powder, the development of non-synthetic powders is insufficient, and accordingly, there is an urgent need for natural powders.

### SUMMARY

The present invention is directed to providing an elastic makeup powder derived from natural inorganic particulates, which uses a natural terial when applied to a cosmetic material, thereby having excellent skin stability, being safe to the human body, having excellent dispersibility and capable of significantly improving the texture of the cosmetic material, and a method of preparing the same.

In addition, the present invention is also directed to providing a method of preparing an elastic makeup powder derived from natural inorganic particulates with high productivity due to use of a natural material, thereby having excellent skin stability and improved physical properties.

To solve the above-described technical problems, the present invention provides a natural material-derived elastic makeup powder 100, which includes:

natural inorganic particulates 110 thereinside; and

an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature, dry-coated on the surface of the inorganic particulates 110.

Preferably, the natural material-derived elastic makeup powder 100 has 3 to 20 parts by weight of the elastic coating layer 120 with respect to 100 parts by weight of the inorganic particulates 110.

The elastic coating layer 120 may further include a plant oil.

Preferably, 1 to 20 parts by weight of the oil is included with respect to 100 parts by weight of the inorganic particulates 110.

Preferably, the natural material-derived elastic makeup powder 100 has a viscoelasticity of 50 to 900 g force/μm² when measured with a rheometer.

In addition, the present invention provides

a natural elastic makeup powder 100, which includes:

natural inorganic particulates 110 thereinside;

an oil coating layer 130 coated on the surface of the inorganic particulates 110, and

an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature; dry-coated on the surface of the oil coating layer 130.

In addition, the present invention provides a makeup powder, which includes:

natural inorganic particulates 110 thereinside;

an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature; dry-coated on the surface of the inorganic particulates; and

an oil coating layer 130 coated on the surface of the elastic coating layer.

In addition, the present invention provides a method of preparing a natural material-derived elastic makeup powder 100, which includes:

S1) preparing natural inorganic particulates 110; and

S2) dry-coating the surface of the inorganic particulates 110 with (a) a triester of glycerin and hydroxy fatty acids or a derivative thereof, which is solid at room temperature; or (b) a glyceryl fatty acid ester or a derivative thereof, which is solid at room temperature.

Preferably, 3 to 20 parts by weight of (a) the triester of glycerin and hydroxy fatty acids or a derivative thereof, which is solid at room temperature; or (b) the glyceryl fatty acid ester or a derivative thereof, which is solid at room temperature, is included with respect to 100 parts by weight of the inorganic particulates 110.

In the dry coating step (S2), the surface may be dry-coated with the above-described material in combination with an oil.

In addition, the present invention provides a method of preparing an elastic makeup powder derived from a natural material, which includes:

S11) preparing natural inorganic particulates 110;

S12) coating the surface of the inorganic particulates 110 with an oil; and

S13) dry-coating the surface of the oil with (a) a triester of glycerin and hydroxy fatty acids or a derivative thereof, which is solid at room temperature; or (b) a glyceryl fatty acid ester or a derivative thereof, which is solid at room temperature.

In addition, the present invention provides a method of preparing an elastic makeup powder derived from a natural material, which includes:

S21) preparing natural inorganic particulates;

S22) dry-coating the surface of the inorganic particulates with (a) a triester of glycerin and hydroxy fatty acids or a derivative thereof, which is solid at room temperature; or (b) a glyceryl fatty acid ester or a derivative thereof, which is solid at room temperature; and

S23) coating the dry-coated surface of the inorganic particulates with an oil.

In addition, the present invention provides a cosmetic material including the natural material-derived makeup powder.

According to the present invention, an elastic makeup powder derived from natural inorganic particulates, which uses a natural material when applied to a cosmetic material, thereby having excellent skin stability, being safe to the human body, having excellent dispersibility and capable of significantly improving the texture of the cosmetic material, and a method of preparing the same can be provided.

In addition, the present invention can provide a method of preparing an elastic makeup powder derived from natural inorganic particulates with high productivity due to the use of a natural-material, thereby having excellent skin stability and improved physical properties.

### DESCRIPTION OF THE DRAWINGS

FIG. 1 is a schematic diagram of an elastic makeup powder derived from natural inorganic particulates according to one embodiment of the present invention.

FIG. 2 is a schematic diagram of an elastic makeup powder derived from natural inorganic particulates according to another embodiment of the present invention.

FIG. 3 is a schematic diagram of an elastic makeup powder derived from natural inorganic particulates according to still another embodiment of the present invention.

FIG. 4 is a schematic diagram of an elastic makeup powder derived from natural inorganic particulates according to one embodiment of the present invention.

FIG. 5 is a schematic diagram of a method of preparing an elastic makeup powder derived from natural inorganic particulates according to another embodiment of the present invention.

FIG. 6 is a schematic diagram of a method of preparing an elastic makeup powder derived from natural inorganic particulates according to still another embodiment of the present invention.

### DETAILED DESCRIPTION

Hereinafter, embodiments of the present invention will be described in detail with reference to the accompanying drawings so that those of ordinary skill in the art can easily carry out the present invention. However, the present invention may be implemented in a variety of different forms, and is not limited to the embodiments described herein. In addition, for clear explanation of the present invention in the drawings, parts that are not related to the description are omitted, and like numerals denote like parts throughout the specification.

Throughout the specification, when one part “includes”a component, it means that it may also include other components, not excluding components unless particularly stated otherwise.

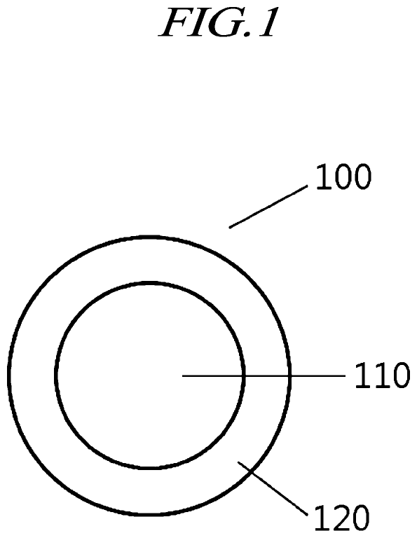

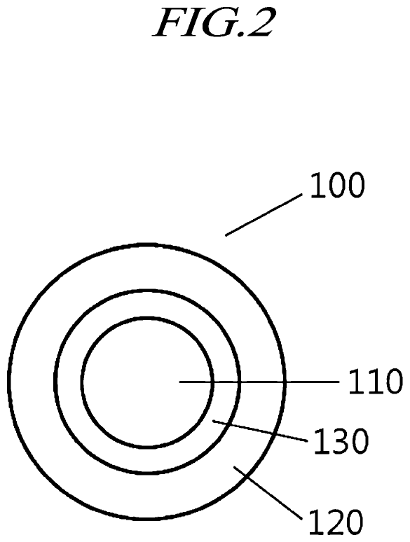

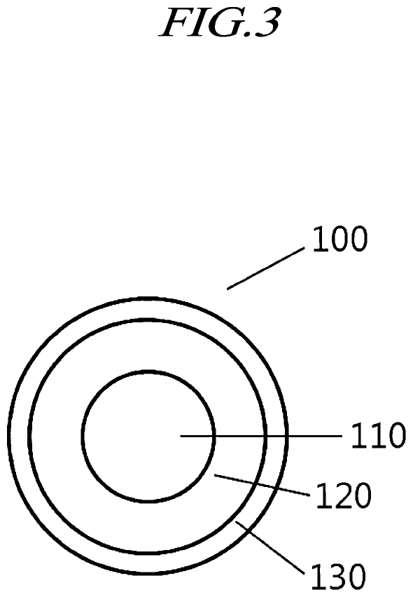

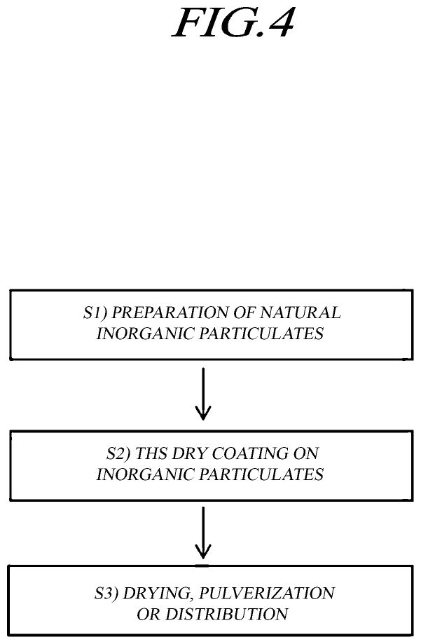

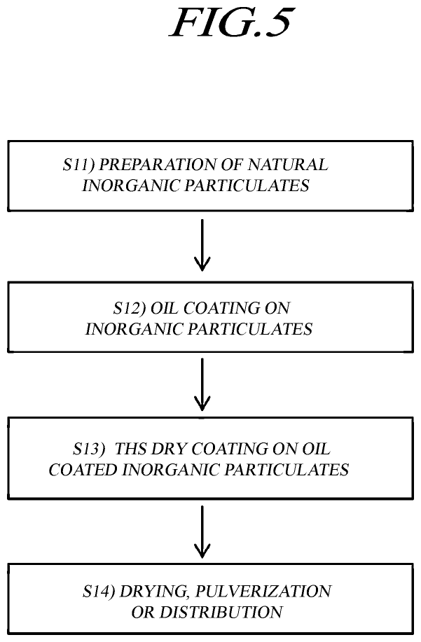

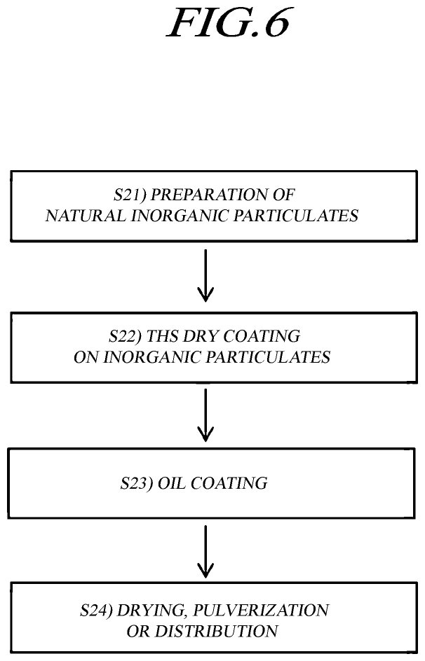

FIG. 1 is a schematic diagram of an elastic makeup powder derived from natural inorganic particulates according to one embodiment of the present invention, FIG. 2 is a schematic diagram of an elastic makeup powder derived from natural inorganic particulates according to another embodiment of the present invention, FIG. 3 is a schematic diagram of an elastic makeup powder derived from natural inorganic particulates according to still another embodiment of the present invention, FIG. 4 is a schematic diagram of an elastic makeup powder derived from natural inorganic particulates according to one embodiment of the present invention, FIG. 5 is a schematic diagram of a method of preparing an elastic makeup powder derived from natural inorganic particulates according to another embodiment of the present invention, and FIG. 6 is a schematic diagram of a method of preparing an elastic makeup powder derived from natural inorganic particulates according to still another embodiment of the present invention.

A natural material-derived elastic makeup powder 100 of the present invention includes

natural inorganic particulates 110 thereinside; and

an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature, dry-coated on the surface of the inorganic particulates 110.

The natural material-derived elastic makeup powder 100 may be prepared by

S1) preparing natural inorganic particulates 110; and

S2) dry-coating the surface of the inorganic particulates 110 with (a) a triester of glycerin and hydroxy fatty acids or a derivative thereof, which is solid at room temperature; or (b) a glyceryl fatty acid ester or a derivative thereof, which is solid at room temperature.

In the present invention, the natural inorganic particulates 110 may be known inorganic particulates that can be used in cosmetics as inorganic particulates derived from a natural material, and as a specific example, silica, talc, sericite, pigment, titanium dioxide, zinc oxide or mica may be used alone or in a combination of two or more thereof. Preferably, the natural inorganic particulates are silica, and the silica used here may or may not have pores.

The particle size of the inorganic particulates 110 may be suitably selected by those of ordinary skill in the art. In one example, the particle size of the inorganic particulates 110 may be 0.1 to 100 μm, but the present invention is not limited thereto.

In the present invention, (a) the triester of glycerin and hydroxy fatty acids or a derivative thereof, which is solid at room temperature; or (b) the glyceryl fatty acid ester or a derivative thereof, which is solid at room temperature, dry-coated on the surface of the inorganic particulates 110 may be known (a) triester of glycerin and hydroxy fatty acids or a derivative thereof, which is solid at room temperature; or (b) glyceryl fatty acid ester or a derivative thereof, which is solid at room temperature, and preferably, they are derived from nature or commercially available. Specifically, (a) the triester of glycerin and hydroxy fatty acids, which is solid at room temperature, may be trihydroxystearin, and (b) the glyceryl fatty acid ester, which is solid at room temperature, may be glyceryl behenate.

In the present invention, (a) the triester of glycerin and hydroxy fatty acids or a derivative thereof, which is solid at room temperature; or (b) the glyceryl fatty acid ester or a derivative thereof, which is solid at room temperature, may be contained at 3 to 20 parts by weight, and preferably, 6 to 10 parts by weight with respect to 100 parts by weight of the inorganic particulates 110. In this case, the prepared elastic powder 100 has elasticity suitable fir cosmetics, and also has excellent stability in cosmetic materials.

In the present invention, for dry coating, a dry coating method conventionally applied in the production of cosmetics may be applied, and as a specific example, the elastic coating layer 120 is formed by dry coating the inorganic particulates 110 with (a) a triester of glycerin and hydroxy fatty acids or a derivative thereof, which is solid at room temperature; or (b) a glyceryl fatty acid ester or a derivative thereof, which is solid at room temperature while stirring at 2000 to 4500 rpm.

As needed, the method of preparing the natural material-derived elastic makeup powder 100 may be performed independently by, after S2, drying the dry-coated inorganic particulates; pulverizing the dry-coated inorganic particulates; and distributing the dry-coated inorganic particulates. The drying, pulverization and distribution steps may be performed independently, or two or more thereof may be performed in combination. In one example, the dry-coated inorganic particulates are formed into the elastic makeup powder 100 through drying, pulverization, or distribution.

The elastic makeup powder 100 according to the present invention has, as shown in FIG. 1, natural inorganic particulates 110 thereinside, and an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature, dry-coated on the surface of the inorganic particulates 110, wherein the elastic coating layer 120 may have a uniform or non-uniform thickness.

The elastic makeup powder 100 according to the present invention may further include an oil.

The oil may be a known oil used in cosmetics, and specifically, a plant oil or plant-derived oil. More specifically, as a plant oil, macadamia nut oil, meadowfoam seed oil and sunflower oil may be used alone or in a combination of two or more thereof, and as a plant-derived oil, caprylic capric triglyceride or dicapryl carbonate may be used alone or in a combination thereof. The plant oil or plant-derived oil may be used in a combination.

Preferably, the oil may be contained at 1 to 20 parts by weight, and preferably, 3 to 10 parts by weight with respect to 100 parts by weight of the inorganic particulates 110. In this case, the prepared elastic powder 100 has elasticity suitable for cosmetics, and also has excellent stability in cosmetic materials.

In the present invention, when the elastic makeup powder 100 includes an oil, the oil may be mixed with (a) the triester of glycerin and hydroxy fatty acids or a derivative thereof, which is solid at room temperature; or (b) the glyceryl fatty acid ester or a derivative thereof, which is solid at room temperature, and subjected to dry, coating, thereby forming an elastic coating layer 120.

In addition, in the present invention, the elastic makeup powder 100, which is a makeup powder with a separate oil coating layer, may include

natural inorganic particulates 110 thereinside;

an oil coating layer 130 coated on the surface of the inorganic particulates 110;

an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature, dry-coated on the surface of the oil coating layer 130.

The natural material-derived elastic makeup powder 100 may be prepared by

S11) preparing natural inorganic particulates 110; and

S12) coating the surface of the inorganic particulates 110 with an oil; and

S13) dry-coating the surface of the oil with (a) a triester of glycerin and hydroxy fatty acids or a derivative thereof, which is solid at room temperature; or (b) a glyceryl fatty acid ester or a derivative thereof, which is solid at room temperature.

A coating method in the step of coating with the oil (S12) may be a known oil coating method, and specifically, dry coating.

In addition, as needed, the method of preparing the natural material-derived elastic makeup powder 100 may be performed independently by, after S13, drying the dry-coated inorganic particulates; pulverizing the dry-coated inorganic particulates; and distributing the dry-coated inorganic particulates. The drying, pulverization and distribution steps may be performed independently, and two or more thereof may be performed in combination. In one example, the dry-coated inorganic particulates are formed into the elastic makeup powder 100 through drying, pulverization, or distribution.

In addition, in the present invention, the elastic makeup powder 100, which is a makeup powder with a separate oil coating layer, may include

natural inorganic particulates 110 thereinside;

an elastic coating layer 120 consisting of (a) a triester of glycerin and hydroxy fatty acids or a derivative thereof, which is solid at room temperature; or (b) a glyceryl fatty acid ester or a derivative thereof, which is solid at room temperature, dry-coated on the surface of the inorganic particulates; and

an oil coating layer 130 coated on the surface of the elastic coating layer 120.

The natural material-derived elastic makeup powder 100 may be prepared by

S21) preparing natural inorganic particulates;

S22) dry-coating the surface of the inorganic particulates with (a) a triester of glycerin and hydroxy fatty acids or a derivative thereof, which is solid at room temperature; or (b) a glyceryl fatty acid ester or a derivative thereof, which is solid at room temperature; and

S23) coating the dry-coated surface of the inorganic particulates with an oil.

As needed, the method of preparing the natural material-derived elastic makeup powder 100 may be performed independently by, after S23, drying the dry-coated inorganic particulates; pulverizing the dry-coated inorganic particulates; and distributing the dry-coated inorganic particulates. The drying, pulverization and distribution steps may be performed independently, and two or more thereof may be performed in combination. In one example, the dry-coated inorganic particulates are formed into the elastic makeup powder 100 through drying, pulverization, or distribution.

The elastic makeup powder 100 according to the present invention may impart excellent texture when applied to a cosmetic material due to its viscoelasticity of 50 to 900 g force/μm² measured with a rheometer (Product Name: MCT-W500-E, Manufacturer: Shimadzu).

In addition, the present invention provides a cosmetic material including the elastic makeup powder 100. There is no limit to formulations of the cosmetic material, and thus all types of formulations that can be applied to cosmetic materials, such as a W/S formulation, a W/O formulation, a powder formulation, an anhydrous formulation, and an O/W formulation, may be applied for various products such as foundations, lipsticks, skincare products, makeup products, or sun-care products.

In the cosmetic material of the present invention, the content of the elastic makeup powder 100 may be arbitrarily adjusted, specifically to 0.1 to 30 wt %.

Hereinafter, the present invention will be described in detail with reference to examples to help in understanding the present invention. However, examples according to the present invention may be modified in a variety of different forms, and it should not be construed that the scope of the present invention is limited to the following examples. The examples of the present invention are provided to more completely explain the present invention to those of ordinary skill in the art.

### Example 1

As natural inorganic particulates, 100 parts by weight of silica was prepared. A makeup powder having a trihydroxystearin elastic coating layer formed on the silica was prepared by inputting 100 parts by weight of the silica to a dry coating apparatus, and performing dry coating while stirring 6 parts by weight of trihydroxystearin at 2000 to 4500 rpm. The elastic makeup powder 100 according to the present invention was able to impart excellent texture when applied to a cosmetic material due to its viscoelasticity of 50 to 900 g force/μm² measured with a rheometer (Product Name: MCT-W500-E, Manufacturer: Shimadzu).

### Example 2

A makeup powder having a glyceryl behenate elastic coating layer formed on silica was prepared by dry coating in the same manner as in Example 1, except that glyceryl behenate was used instead of trihydroxystearin. The makeup powder was able to give excellent texture when applied to a cosmetic material due to its viscoelasticity of 50 to 900 g force/μm² measured with a rheometer (Product Name: MCT-W500-E, Manufacturer: Shimadzu).

### Example 3

A makeup powder having an elastic coating layer was prepared by dry coating in the same manner as in Example 1, except that 10 parts by weight of trihydroxystearin was used. The makeup powder was able to give excellent texture when applied to a cosmetic material due to its viscoelasticity of 50 to 900 g force/μm² measured with a rheometer (Product Name: MCT-W500-E, Manufacturer: Shimadzu).

### Example 4

A makeup powder having an elastic coating layer was prepared by dry coating in the same manner as in Example 1, except that 5 parts by weight of a plant oil was further mixed as an oil. The makeup powder was able to give excellent texture when applied to a cosmetic material due to its viscoelasticity of 50 to 900 g force/μm² measured with a rheometer (Product Name: MCT-W500-E, Manufacturer: Shimadzu).

### Example 5

A makeup powder having an inorganic particulate layer, an oil layer, and an elastic coating layer was prepared by dry coating in the same manner as in Example 1, except that inorganic particulates were first coated with 5 parts by weight of a plant oil as an oil and then dry-coated with trihydroxystearin. The makeup powder was able to give excellent texture when applied to a cosmetic material due to its viscoelasticity of 50 to 900 g force/μm² measured with a rheometer (Product Name: MCT-W500-E, Manufacturer: Shimadzu).

### Example 6

A makeup powder having an inorganic particulate layer, an elastic coating layer, and an oil layer was prepared by dry coating in the same manner as in Example 1, except that inorganic particulates were dry-coated with trihydroxystearin and then dry-coated with 5 parts by weight of a plant oil as an oil. The makeup powder was able to give excellent texture when applied to a cosmetic material due to its viscoelasticity of 50 to 900 g force/μm² measured with a rheometer (Product Name: MCT-W500-E, Manufacturer: Shimadzu).

### Reference Example 1

A makeup powder having an elastic coating layer was prepared by dry coating in the same manner as in Example 1, except that 1 part by weight of trihydroxystearin was used. It was able to be confirmed that the makeup powder had low elasticity of less than 50 g force/μm² measured with a rheometer (Product Name: MCT-W500-E, Manufacturer: Shimadzu) when applied to a cosmetic material.

### Reference Example 2

A makeup powder having an elastic coating layer was prepared by dry coating in the same manner as in Example 1, except that 30 parts by weight of trihydroxystearin was used. The makeup powder showed agglomeration, and it was confirmed that the stability was lowered when applied to a cosmetic material.

While the present invention has been described in detail with reference to exemplary embodiments of the present invention, it should be understood by those of ordinary skill in the art that the scope of the present invention is not limited thereto and various forms of modification and alternation based on the fundamental ideas of the present invention defined by the accompanying claims are also included within the scope of the present invention.

### INDUSTRIAL APPLICABILITY

According to the present invention, an elastic makeup powder derived from natural inorganic particulates, which uses a natural material when applied to a cosmetic material, thereby having excellent skin stability, being safe to the human body, having excellent dispersibility and capable of significantly improving the texture of the cosmetic material, and a method of preparing the same can be provided.

In addition, the present invention can provide a preparation method for preparing an elastic makeup powder derived from natural inorganic particulates with high productivity due to the use of a natural material, thereby having excellent skin stability and improved physical properties.

1. An elastic makeup powder derived from a natural material, comprising:
natural inorganic particulates thereinside; and
an elastic coating layer consisting of (a) a triester of glycerin and hydroxy fatty acids or a derivative thereof, which is solid at room temperature; or (b) a glyceryl fatty acid ester or a derivative thereof, which is solid at room temperature, dry-coated on the surface of the inorganic particulates.

2. The powder of claim 1, wherein the elastic coating layer is contained at 3 to 20 parts by weight with respect to 100 parts by weight of the inorganic particulates.

3. The powder of claim 1, wherein the elastic coating layer further comprises an oil.

4. The powder of claim 3, wherein the oil is contained at 1 to 20 parts by weight with respect to 100 parts by weight of the inorganic particulates.

5. The powder of claim 3, further comprising:
natural inorganic particulates thereinside;
an oil coating layer coated on the surface of the inorganic particulates; and
an elastic coating layer consisting of (a) a triester of glycerin and hydroxy fatty acids or a derivative thereof, which is solid at room temperature; or (b) a glyceryl fatty acid ester or a derivative thereof, which is solid at room temperature; dry-coated on the surface of the oil coating layer.

6. The powder of claim 3, further comprising:
natural inorganic particulates thereinside;
an elastic coating layer consisting of (a) a triester of glycerin and hydroxy fatty acids or a derivative thereof, which is solid at room temperature; or (b) a glyceryl fatty acid ester or a derivative thereof, which is solid at room temperature; dry-coated on the surface of the inorganic particulates; and
an oil coating layer coated on the elastic coating layer.

7. A method of preparing an elastic makeup powder derived from a natural material, comprising:
S1) preparing natural inorganic particulates; and
S2) dry-coating the surface of the inorganic particulates with (a) a triester of glycerin and hydroxy fatty acids or a derivative thereof, which is solid at room temperature: or (b) a glyceryl fatty acid ester or a derivative thereof, which is solid at room temperature.

8. The method of claim 7, wherein the elastic coating layer is contained at 3 to 20 parts by weight with respect to 100 parts by weight of the inorganic particulates.

9. The method of claim 7, further comprising:
S11) preparing natural inorganic particulates;
S12) coating the surface of the inorganic particulates with an oil; and
S13) dry-coating the surface of the oil with (a) a triester of glycerin and hydroxy fatty acids or a derivative thereof, which is solid at room temperature; or (b) a glyceryl fatty acid ester or a derivative thereof, which is solid at room temperature.

10. The method of claim 7, further comprising:
S21) preparing natural inorganic particulates;
S22) dry-coating the surface of the inorganic particulates with (a) a triester of glycerin and hydroxy fatty acids or a derivative thereof, which is solid at room temperature; or (b) a glyceryl fatty acid ester or a derivative thereof, which is solid at room temperature; and;
S23) coating the dry-coated surface of the inorganic particulates with an oil.

11. A cosmetic material comprising the natural material-derived makeup powder of claim 1.
